(19) United States
(12) Patent Application Publication
(10) Pub. No.: US 2020/0323448 A1
(43) Pub. Date: Oct. 15, 2020

(54) System of Determining Physiological State

(71) Applicant: FaceHeart Inc., Hsinchu City (TW)

(72) Inventors: Tsuey-Huey Shiue, Hsinchu City (TW); Peng-Wen Lai, Tainan City (TW); Kuan-Hung Chen, Hsinchu City (TW); Yin-Cheng Tsai, Taoyuan City (TW)

(21) Appl. No.: 16/452,470

(22) Filed: Jun. 25, 2019

(30) Foreign Application Priority Data
Apr. 12, 2019 (TW) 108112901

(51) Int. Cl. A61B 5/024; G16H 50/20; A61B 5/1455

(52) U.S. Cl. CPC A61B 5/02405; G16H 50/20; G16H 10/60; A61B 5/1455; A61B 5/02416

(57) ABSTRACT

A system of determining physiological state includes a physiological measuring device configured to measure at least one physiological parameter of a user, a user interface configured to receive at least one physiological information of the user, and a processing unit coupled to the physiological measuring device and the user interface, and configured to determine at least one normal threshold and at least one abnormal threshold of the at least one physiological parameter according to the at least one physiological information, and determine a physiological state of the user is normal or abnormal according to the at least one physiological parameter, the at least one normal threshold and the at least one abnormal threshold.

### BACKGROUND OF THE INVENTION

### 1. Field of the Invention

The present invention relates to a system of determining physiological state, and more particularly, to a system of determining physiological state capable of determining normal and abnormal thresholds corresponding to a physiological parameter of a user.

### 2. Description of the Prior Art

How to determine a physiological state of a user to be normal or abnormal has become a new topic of the industry. It could be mistakenly reflect the physiological state of the user to be normal or abnormal only based on measurement results from a physiological measuring device (e.g., a heart rate, a blood pressure, a respiratory rate, and the like), because the physiological state of the user is also related to a personal body condition (e.g., gender, age, body mass index, and the like) , a medical history record, a medicine record and a fatigue index. Therefore, there might be controversy when determining the physiological state of the user to be normal or abnormal with insufficient data.

In practice, for a driver requiring high attention during driving, it is important to take an examination of physiological state before hitting the road, which ensures driving safety. Therefore, how to precisely determine the physiological state of the user is normal or abnormal to ensure driving safety is indeed a critical topic in the industry.

### SUMMARY OF THE INVENTION

It is therefore an objective of the present invention to provide a system of determining physiological state configured to precisely determine the physiological state of the user is normal or abnormal.

The present invention discloses a system of determining physiological state includes a physiological measuring device configured to measure at least one physiological parameter of a user, wherein the at least one physiological parameter comprises at least one of a heart rate, a heart rate variance, a facial expression feature, a blood pressure, a respiratory rate and a blood oxygen concentration; a user interface configured to receive at least one physiological information of the user, wherein the at least one physiological information comprises at least one of a medical history, a medicine record, an alcohol consumption and a fatigue index; and a processing unit coupled to the physiological measuring device and the user interface, and configured to determine at least one normal threshold and at least one abnormal threshold corresponding to the at least one physiological parameter of the user according to the at least one physiological information; and determine a physiological state of the user is normal or abnormal according to the at least one physiological parameter, the at least one normal threshold and the at least one abnormal threshold.

The system of determining physiological state of the present invention may utilize the processing unit to adjust algorithms for determining the normal threshold and the abnormal threshold based on the physiological information of the user (e.g., medical history record, medicine record, alcohol consumption and fatigue index) and the physiological parameter of the user (heart rate, heart rate variance, facial expression feature, blood pressure, respiratory rate and blood oxygen concentration), so as to precisely determine the physiological state of the user is normal or abnormal.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a functional block diagram of a system of determining physiological state according to an embodiment of the present invention.

FIG. 2 is a schematic diagram of the physiological measuring device in FIG. 1 according to an embodiment of the present invention.

### DETAILED DESCRIPTION

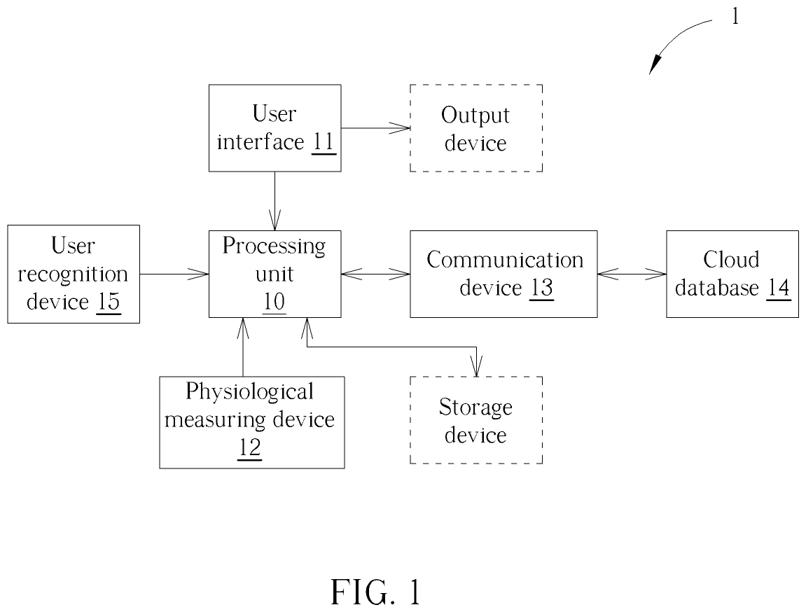

FIG. 1 is a functional block diagram of a system of determining physiological state 1 according to an embodiment of the present invention. The system of determining physiological state 1 includes a processing unit 10, a user interface 11, a physiological measuring device 12, a communication device 13, a cloud database l4and a user recognition device 15.

The physiological measuring device 12 is coupled to the processing unit 10, and configured to measure at least one physiological parameter of the user, wherein the at least one physiological parameter includes at least one of a heart rate, a heart rate variance, a facial expression feature, a blood pressure, a respiratory rate and a blood oxygen concentration. The user interface 11 is coupled to the processing unit 10, and configured to receive the at least one physiological information of the user, wherein the at least one physiological information includes at least one of a medical history record, a medicine record, an alcohol consumption and a fatigue index.

The processing unit 10 is coupled to the physiological measuring device 12 and the user interface 11, and configured to determine a normal threshold and an abnormal threshold corresponding to the physiological parameter of the user according to the at least one physiological parameter and the at least one physiological information. The processing unit 10 is further configured to determine a physiological state of the user is normal or abnormal according to the at least one physiological parameter, the normal threshold and the abnormal threshold.

In one embodiment, the processing unit 10 may be a server or a computer, and include a system unit (including a control unit, an arithmetic logic unit and a main memory unit), an external storage device, an input unit and an output unit. The control unit is configured to control the arithmetic logic unit to access the main memory unit and the external storage device to perform computation. The input unit is configured to receive the physiological parameter and the physiological information, and the output unit is configured to output the physiological state.

In detail, if the medical history record of the user indicates, for example, hypertension, heart disease, anemia, sleep apnea, lung disease, diabetes, mental illness, gout, epilepsy, stroke and the like, the physiological parameter (including heart rate, heart beat interval, blood pressure, respiratory rate, blood oxygen concentration, and the like) of the user may change after the user has taken the medicine. After the processing unit 10 analyzed the medical history record and the medicine record, the normal threshold and the abnormal threshold corresponding to the physiological parameter may be determined. For example, a normal threshold of a blood pressure without hypertension is classified into a systolic blood pressure less than 120 mmHg and a diastolic blood pressure less than 80 mmHg; while a normal threshold of a blood pressure with hypertension is classified into a systolic blood pressure from 120 to 139 mmHg and a diastolic blood pressure from 80 to 89 mmHg. Therefore, for the blood pressure of the user having hypertension, the physiological state of the user is determined to be normal when the physiological measuring device 12 measures a systolic blood pressure from 120 to 139 mmHg and a diastolic blood pressure from 80 to 89 mmHg; the physiological state of the user is determined to be abnormal when physiological measuring device 12 measures the systolic blood pressure is greater than 139 mmHg and the diastolic blood pressure is greater than 89 mmHg.

From another perspective view, since the “normal” blood pressure for users having different diseases is different, the system of determining physiological state 1 of the present invention is able to avoid the blood pressure of all users from corresponding to a single normal threshold, so as to adapt to multiple embodiment types.

As a result, the system of determining physiological state 1 of the present invention may utilize the processing unit 10 to adjust algorithms for determining the normal threshold and the abnormal threshold based on the physiological information of the user (e.g., medical history record, medicine record, alcohol consumption and fatigue index) and the physiological parameter of the user (heart rate, heart rate variance, facial expression feature, blood pressure, respiratory rate and blood oxygen concentration) , so as to precisely determine the physiological state of the user is normal or abnormal.

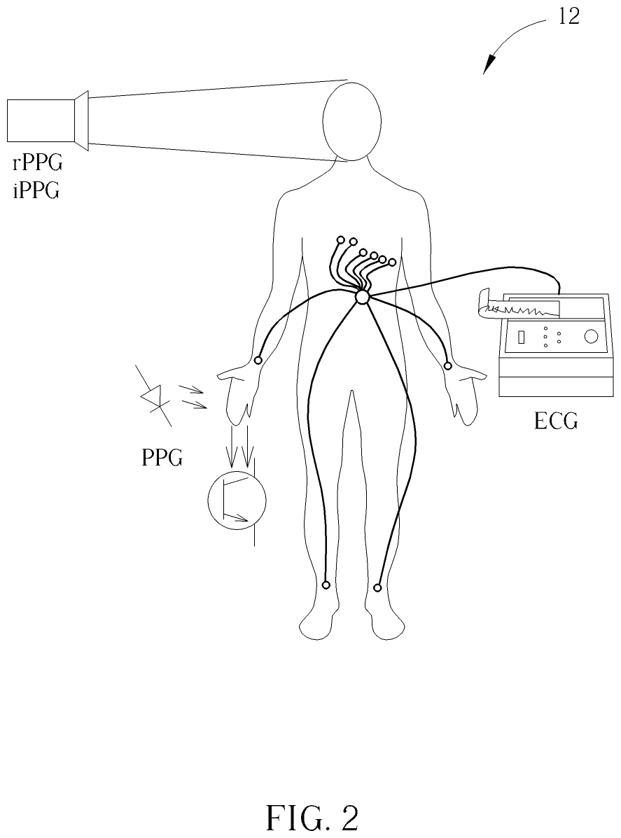

FIG. 2 is a schematic diagram of the physiological measuring device 12 according to an embodiment of the present invention. In one embodiment, the physiological measuring device 12 is an electrocardiograph (ECG) , and configured to capture and record electrophysiological activities of a heart under unit time through electrodes attached on the chest skin of the subject, so as to generate an electrocardiograph. In one embodiment, the physiological measuring device is a signal conversion module configured to convert a target area image of the user into a photoplethysmography (PPG), a remotephotoplethysmography (rPPG) or a imaging PPG (iPPG), wherein the photoplethysmography is used for predicting blood pressure (e.g., pulse pressure, diastolic blood pressure, systolic blood pressure, and so on) .

Before the physiological measuring device 12 perform measurement, the physiological information of the user may be inputted through multiple user interfaces 11, e.g. , the user may use a personal computer (with handwriting or keyboard, mouse clicking icons) , a tablet computer, a smart phone, a wearable device, a biological chip, a card reader and the like, to input the physiological information of the user to the processing unit 10. The processing unit 10 may connect to a build-in or external storage device configured to store the physiological information, the physiological parameter and the corresponding physiological determination result.

In one embodiment, personal information of the user is permitted to input to the system of determining physiological state 1 only when an agreement of the user is obtained, and the personal information of user should be encrypted to protect the personal information of user. Accordingly, the user recognition device 15 may be configured to encrypt or decrypt the personal information or an account of user to protect the personal information of user, wherein user recognition device 15 is configured to control read and write operations of the processing unit 10 to the user interface 11 through a security technology of a fingerprint recognition, a face recognition, a digital number password, a graphic password. In one embodiment, the user interface 11 may integrate with or connect to an output device configured to output the physiological determination result to the user, wherein the output device may be a display device, a speaker, a light, a beeper, and the like.

The communication device 13 is coupled to the user interface 11 and the processing unit 10, and configured to connect the processing unit 10 to the cloud database 14 through the Internet, a universal serial bus, WiFi, a third/fourth/fifth generation mobile communication system, Bluetooth, near field communication or a network communication device. The cloud database 14 may be an insurance company database, a personal cloud database, and the like.

To sum up, the system of determining physiological state of the present invention may utilize the processing unit to adjust algorithms for determining the normal threshold and the abnormal threshold based on the physiological information of the user (e.g., medical history record, medicine record, alcohol consumption and fatigue index) and the physiological parameter of the user (heart rate, heart rate variance, facial expression feature, blood pressure, respiratory rate and blood oxygen concentration) , so as to precisely determine the physiological state of the user is normal or abnormal.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A system of determining physiological state, comprising:
a physiological measuring device configured to measure at least one physiological parameter of a user, wherein the at least one physiological parameter comprises at least one of a heart rate, a heart rate variance, a facial expression feature, a blood pressure, a respiratory rate and a blood oxygen concentration;
a user interface configured to receive at least one physiological information of the user, wherein the at least one physiological information comprises at least one of a medical history, a medicine record, an alcohol consumption and a fatigue index; and
a processing unit coupled to the physiological measuring device and the user interface, and configured to determine at least one normal threshold and at least one abnormal threshold corresponding to the at least one physiological parameter of the user according to the at least one physiological information; and determine a physiological state of the user is normal or abnormal according to the at least one physiological parameter, the at least one normal threshold and the at least one abnormal threshold.

2. The system of determining physiological state of claim 1, wherein the physiological measuring device is a electrocardiograph, and configured to capture and record electrophysiological activities of a heart under unit time through electrodes attached on the chest skin of the user, to generate the at least one physiological parameter.

3. The system of determining physiological state of claim 1, wherein the physiological measuring device is a signal conversion module configured to convert a target area image of the user into a Photoplethysmography, a RemotePhotoplethysmography or an imaging PPG.

4. The system of determining physiological state of claim 1, further comprising a cloud database coupled to the user interface and the processing unit, and configured to store the at least one physiological parameter, the at least one physiological information, the at least one normal threshold and the at least one abnormal threshold.

5. The system of determining physiological state of claim 4, further comprising a communication device coupled to the processing unit, and configured to connect the processing unit to the cloud database through the Internet, a universal serial bus, WiFi, a third/fourth/fifth generation mobile communication system, Bluetooth, near field communication or a network communication device.

6. The system of determining physiological state of claim 1, further comprising a user recognition device coupled to the user interface and the processing unit, and configured to control read and write operations of the processing unit to the user interface through a security technology of a fingerprint recognition, a face recognition, a digital number password, a graphic password.

7. The system of determining physiological state of claim 1, wherein the processing unit determines that the physiological state of the user is normal when the at least one physiological parameter is classified into the at least one normal threshold; and the processing unit determines that the physiological state of the user is abnormal when the at least one physiological parameter is classified into the at least one abnormal threshold.
